(19) United States
(12) Patent Application Publication
(10) Pub. No.: US 2009/0250526 A1
(43) Pub. Date: Oct. 8, 2009

(54) Fragrance Device

(76) Inventors: Colin Litten-Brown, Berkshire (GB); Guy Edward Naish, Oxfordshire (GB)

Correspondence Address: CURATOLO SIDOTI CO., LPA, 24500 CENTER RIDGE ROAD, SUITE 280, CLEVELAND, OH, 44145, US

(21) Appl. No.: 12/515,116

(22) PCT Filed: Nov. 14, 2007
(86) PCT No.: PCT/CH07/00573

§ 371 Date: Jun. 10, 2009

(30) Foreign Application Priority Data
Nov. 16, 2006 (GB) 0622876.1

(51) Int. Cl. A61L 9/04

(52) U.S. Cl. 239/6; 239/57

(57) ABSTRACT

A device adapted to disseminate one of a plurality of volatile liquids into an enclosed atmosphere, the device comprising (i) an enclosure (1) within which is defined a plurality of separate compartments (3,4), each compartment having at least one inlet and at least one outlet port (10, 9), and within the compartment, a source of volatile liquid (5), (ii) a source of forced air current external to the compartments and adapted to blow air through them into the atmosphere; characterised in that the device additionally comprises automatic liquid regulating means in the form of paired port closures (11, 12) for both inlet and outlet ports of each compartment, slidable between a number of inlet and outlet port-closing rest positions equal to the number of compartments, such that, in any rest position, the inlet and outlet ports of only one compartment are open.

This invention relates to devices for disseminating volatile liquid into an enclosed atmosphere, especially fragrance into the interior of a motor vehicle.

Air fresheners for motor vehicles are well known to the art, and many types are available on the market. All function acceptably well, but all have a common problem, that of “habituation”, that is, the fact that the person smelling the fragrance becomes used to it, and therefore no longer notices it. Ways of overcoming this in domestic situations have been proposed, most of these involving a plurality of fragrances (or other desirable volatile liquids, such as malodour counteractants, insecticides, fungicides, disinfectants and the like), and some means of selecting a particular liquid for dissemination. One example of this is UK patent application GB 2 401 047, in which is described a shutter mechanism, which moves a shutter to block one of two possible airflow paths to two fragrance containers, such that only one fragrance at a time is emitted. However, such apparatus, while effective, are relatively large and complex (and therefore relatively expensive). They are thus not suitable for use in motor vehicles, where simplicity and cheapness are desired.

It has now been found that these problems can be partially or completely overcome by a simple device. There is therefore provided a device adapted to disseminate one of a plurality of volatile liquids into an enclosed atmosphere, the device comprising

(i) an enclosure within which is defined a plurality of separate compartments, each compartment having at least one inlet and at least one outlet port, and within the compartment, a source of volatile liquid,

(ii) a source of forced air current external to the compartments and adapted to blow air through them into the atmosphere;

characterised in that the device additionally comprises automatic liquid regulating means in the form of paired port closures for both inlet and outlet ports of each compartment, slidable between a number of inlet and outlet port-closing rest positions equal to the number of compartments, such that, in any rest position, the inlet and outlet ports of only one compartment are open.

There is additionally provided a method of disseminating one of a plurality of volatile liquids into an atmosphere, comprising

(i) providing a plurality of separate compartments, each compartment having at least one inlet and at least one outlet port, and within the compartment a source of volatile liquid,

(ii) causing a source of forced air current external to the compartments to blow air into the atmosphere through the inlet and outlet ports of one compartment at a time;

characterised in that the forced air current is caused to blow through only one compartment at a time, by closing the inlet and outlet ports of all but one compartment by means of paired port closures for both inlet and outlet ports of each compartment, slidable between a number of inlet and outlet port-closing rest positions equal to the number of compartments, and configured such that, in any rest position, the inlet and outlet ports of only one compartment are open.

Although the following discussion will concentrate on a particular embodiment, an air freshener for use in a motor vehicle, it will be readily appreciated that the device is not limited to this use, and devices functioning on this principle can be used in many domestic, industrial and institutional locations for the dissemination of a variety of desirable liquids.

The enclosure with its separate compartments for each liquid may be any such enclosure made of any suitable material. In the case of a car air freshener, the enclosure may be a plastics moulding, which has the advantage of cheapness and choice of colour and shape, but the enclosure can equally be metal, wood, ceramic or any other suitable material. The number of compartments will depend on the number of liquids desired. In the case of motor vehicle air fresheners, two is generally sufficient. More is possible, but the device increases in size, complexity and inconvenience with increasing number of liquids. However, this is not so much a handicap in a domestic/institutional situation, and three or more liquids may be easily provided in such a case.

The liquids for dissemination are provided in the enclosure in individual compartments, which are isolated from each other and which communicate with the atmosphere via at least one exit port in each compartment. The liquid may be provided by any convenient means. In principle, any technology used in room air fresheners can also be used here. For example, the liquid may be provided by a liquid-loaded gel, or a porous wick with one end in a reservoir of liquid or a liquid sealed in a reservoir by a semi-permeable membrane.

The liquid is disseminated by means of forced air current blowing into the inlet port and conveying liquid-bearing air into the atmosphere through the outlet port. This many be achieved by having a fan or blower, typically the kind that is used in laptop computers, mounted within the enclosure and operated by batteries or electricity provided by the vehicle electrical system, for example, by being wired into the vehicle or by being plugged into the cigarette lighter. Another possibility is the use the vehicle's own ventilation system to provide the air current. In such a case, an enclosure may be adapted for attachment to the ventilation ducts of the vehicle, and provided with inlet ports adjacent to the ventilation ducts, such that the air current can blow through the enclosure and out through the outlet port.

The automatic liquid regulating means, which ensures that only one liquid at a time is disseminated, has the form of slidable paired port closures, configured such that only one compartment can communicate with the atmosphere at any one time. By “automatic” is meant that the change from the dissemination of one liquid to another is not achieved manually. This aspect will be described further hereinunder. By “paired” is meant that the closures for the inlet and outlet ports of any given compartment move as one, and not individually, such that the inlet and outlet ports of any given compartment are either both open or both closed. By “slidable” is meant the sliding movement of one surface over another, either linearly or rotary.

In a particular embodiment, the slidable port closures form part of a single slidable entity that works on all ports in all compartments. Such an entity may be made up of a number of individual parts, but they form part of and move as a single entity. The single entity shall hereinafter referred to as “the closure component” and further discussion of the device will be in the context of this arrangement, which is particularly cheap and efficient, especially for automotive air fresheners. However, it should be borne in mind that it is possible to have two or more paired port enclosures working as independent entities, but still functioning to close off all compartments but one. Such an arrangement is especially advantageous when the number of compartments involved is three or more.

The closure component with its port closures is slidably movable between a number of rest positions equal to the number of compartments, The rest positions may be defined by any convenient means, and this will be further discussed hereinunder.

The requirement that the closures close the inlet and outlet ports of all compartments but one in any rest position means that the arrangement of the closures and the various ports must be such that the position of a pair of closures in any rest position will ensure that the ports of one compartment are open while those of the other compartment(s) are closed. In the case of two compartments, typical of a motor vehicle air freshener, this is relatively easily achieved; it becomes more complex with three or more compartments, but it can be achieved and the skilled person will readily be able to provide this through the exercise of the ordinary skill of the art.

A typical closure component has the form of a pair of combs, one for the inlet ports and one for the outlet ports, these combs having a small number of relatively wide teeth, which form the port closures. The closure component is mounted in or on the enclosure in such a way that it can slide between various rest positions, and the teeth are placed on the combs such that, in any rest position, the teeth are in front of and block the port(s) of all but one compartment(s). In practice, the simplest, most reliable and most cost-effective arrangement is to arrange the combs in vertical, inverted positions, such that the sliding will be linear and sideways. However, with an actuation means of sufficient strength and robustness, it may also slide vertically. There are other ways of realising this; for example, the port closure component may be rotary instead of linearly movable, and may comprise, for example, a pair of discs with suitable apertures working in combination with a suitably ported enclosure. These and other variations are all within the skill of the art.

The movement of the closure component may be actuated by any convenient means. For example, in the case of a two-compartment vehicle air freshener, the actuation may be performed by an electrical solenoid. In such a case, the two rest positions of the closure component are defined by the two positions of the solenoid. Another way of providing a two-position change is the use of SMA (shape memory alloy), sometimes known as “memory metal” or “smart metal”. This is an alloy that can change its shape, but that “remembers” the original shape and can revert to it when a shape-changing stimulus is removed. This stimulus may be, for example, the application of heat or an electrical current, the latter being more appropriate for small devices such as vehicle air fresheners. Such materials are well-known items of commerce, one example being Nitinol™ of NDC Materials Group.

Where three or more liquids (and therefore compartments) are required, a more complicated position changer is needed. Such a device may easily be provided using the normal technology available to the art, for example, by making the paired port closures movable independently of each other and programming them such that only one compartment at a time is open to the forced air flow. While easily achievable in practice, such devices are bigger and more complex and are generally only desirable in stationary applications, such as domestic, industrial and institutional premises.

The ancillary electrics/electronics needed to actuate such a port closing means are well known to the art and can be easily implemented. They will be powered by on-board batteries or by taking power from a source of power, in the case of a vehicle air freshener directly by being wired into the vehicle or from the cigarette lighter. The automatic functioning of the port closure to stop the dissemination of one liquid and start the dissemination of another is achieved by any convenient means. A typical means comprises a timing mechanism that determines when changes are to be made and which cooperates with the ancillary electrics/electronics to cause the change when necessary. This timing mechanism may be fixed, or it may be variable, allowing a user to set a desired timing.

Thus, in the case of a solenoid-actuated device, the timing mechanism would allow a particular liquid to be emitted for a set time, at which point it would cause the solenoid to move to its other position. The solenoid movement would cause the port closure to move, such that it closes the ports of the previously emitting compartment and simultaneously opens the ports of a previously closed compartment, causing it to begin dissemination.

The devices of this invention are cheap and simple to make from readily-available components, and are efficient and reliable in operation.

The device is further described with reference to the drawings, which depict preferred embodiments and which are not intended to be in any way limiting on the scope thereof. The device depicted in the drawings is an air freshener for a motor vehicle, adapted to utilise the airflow of the vehicle's own ventilation system.

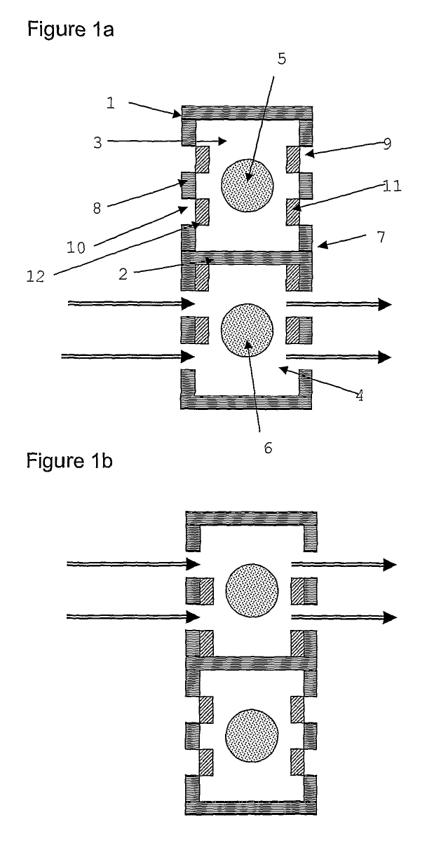

FIGS. 1a and 1b represent schematic plan views of a horizontal cross-section of an air freshener

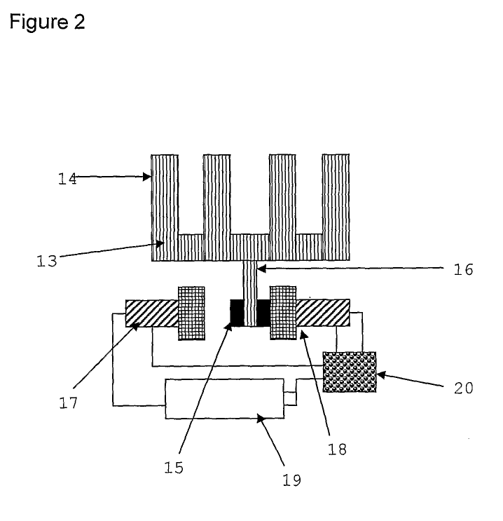

FIG. 2 represents a schematic front elevation view of a port closing mechanism.

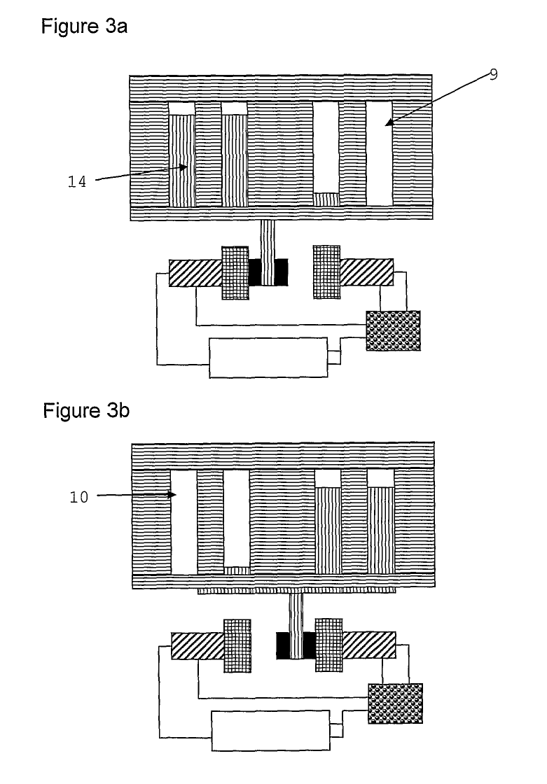

FIGS. 3a and 3b represent schematic front elevation views of the port closing mechanism of FIG. 2 in operation.

In FIG. 1a, the device comprises an enclosure 1 divided by an internal wall 2 into two compartments 3 and 4. Within each compartment is a source of volatile liquid, in this case, a porous wick, 5, 6, which is immersed at its lower end in a volatile liquid in a reservoir (not shown). The enclosure has a front wall 7 and a rear wall 8, the rear wall adapted to abut a ventilation duct of a motor vehicle. Each wall has a series of ports, outlet ports 9 and inlet ports 10, two on each side per compartment in this particular case, through which air from the vehicle ventilation duct can blow when the ports are open.

The ports on both sides may be closed or opened by a pair of port closures 11, 12, which form part of a closure component 13 (FIG. 2). This has the form of a pair of vertically-mounted inverted combs (only one shown in FIG. 2) with four broad teeth 14, each corresponding to a port and arranged such that, when the teeth are positioned to close the ports of compartment 3, the corresponding ports of compartment 4 are open, and vice versa. The relative widths of the teeth and the ports are such that, when the port closure component is moved, the ports of one compartment are either completely closed or completely open, the widths of those portions of the front or rear walls between the ports being at least equal to the widths of the teeth.

Movement of the port closure component is caused by a solenoid mechanism. This consists of a horizontally-slidable ferromagnetic element 15, typically a steel bar, to which the port closure component is fixed by means of a shaft 16 attached to the bar. The two positions of the bar (and therefore those of the teeth) are determined by the placing of two electromagnets 17,18. These are actuated by a power source 19 and a control/timer circuit 20. Thus, when the bar 15 moves, the closure component and its associated port closures also move.

The operation of the ports is illustrated in FIG. 3. The actuation of the electromagnet 17 by the control circuit 20 causes the bar 15 to move to the left (in FIG. 3). This causes the outlet and inlet ports 9, 10 in compartment 4 to be blocked, and those of compartment 3 to be open, thus allowing air to blow through, as shown by the arrows (FIG. 1b), conveying fragrance out of compartment 3 and into the interior of the vehicle When the other electromagnet 18 is actuated, the bar 15 moves to the right, such that the front and rear ports 9,10 of compartment 3 are now open and the airflow can now convey fragrance from compartment 4 (FIG. 1a).

The skilled person will appreciate that there are many possible variations and embodiments all of which are realisable by use of the ordinary skill of the art and all of which are within the scope of this invention.

1. A device adapted to disseminate one of a plurality of volatile liquids into an enclosed atmosphere, the device comprising
(i) an enclosure within which is defined a plurality of separate compartments, each compartment having at least one inlet and at least one outlet port, and within the compartment, a source of volatile liquid,
(ii) a source of forced air current external to the compartments and adapted to blow air through them into the atmosphere;
characterised in that the device additionally comprises automatic liquid regulating means in the form of paired port closures for both inlet and outlet ports of each compartment, slidable between a number of inlet and outlet port-closing rest positions equal to the number of compartments, such that, in any rest position, the inlet and outlet ports of only one compartment are open.

2. A device according to claim 1, in which the port closures for all compartments form part of a single slidable entity comprising a closure component.

3. A device according to claim 2, in which the closure component has the form of a pair of combs with a small number of relatively wide teeth, adapted to slide together linearly, the teeth being placed on the comb such that, in any rest position, they block the inlet and outlet port(s) of all but one compartment(s).

4. A device according to claim 1, in which the closure component comprises a pair of rotatable discs with suitable inlet and outlet ports.

5. A device according to claim 1, in which the forced air current is provided by the ventilation ducts of an automotive ventilation system to which the device is attached.

6. A device according to claim 1, in which the port opening and closing is actuated by a solenoid.

7. A device according to claim 1, in which the port opening and closing is actuated by a shape memory alloy.

8. A device according to claim 1, additionally comprising a timing mechanism.

9. A method of disseminating one of a plurality of volatile liquids into an atmosphere, comprising
(i) providing a plurality of separate compartments, each compartment having at least one inlet and at least one outlet port, and within the compartment a source of volatile liquid,
(ii) causing a source of forced air current external to the compartments to blow air into the atmosphere through the inlet and outlet ports of one compartment at a time;
characterised in that the forced air current is caused to blow through only one compartment at a time, by closing the inlet and outlet ports of all but one compartment by means of paired port closures for both inlet and outlet ports of each compartment, slidable between a number of inlet and outlet port-closing rest positions equal to the number of compartments, and configured such that, in any rest position, the inlet and outlet ports of only one compartment are open.
